(19) United States
(12) Patent Application Publication
(10) Pub. No.: US 2006/0253194 A1
(43) Pub. Date: Nov. 9, 2006

(54) DEVICES AND METHODS FOR DISPLACING BIOLOGICAL FLUIDS INCORPORATING STACKED DISC IMPELLER SYSTEMS

(75) Inventor: Daniel Christopher Dial, Shelton, WA (US)

Correspondence Address: SPECKMAN LAW GROUP PLLC, 1201 THIRD AVENUE, SUITE 330, SEATTLE, WA, 98101, US

(73) Assignee: DIAL DISCOVERIES, LLC, Olympia, WA (US)

(21) Appl. No.: 11/381,735

(22) Filed: May 4, 2006

Related U.S. Application Data
(60) Provisional application No. 60/678,070, filed on May 5, 2005.

(51) Int. Cl. A61M 1/10

(52) U.S. Cl. 623/3.13

(57) ABSTRACT

A pump system for moving biological fluids that comprises two stacked disc impeller systems that are magnetically driven by a central driving motor is provided. The pump system may be employed either ex vivo or in vivo.

### CROSS-REFERENCE TO RELATED APPLICATIONS

This application claims priority to U.S. Provisional Patent Application No. 60/678,070, filed May 5, 2005, the disclosure of which is hereby incorporated by reference.

### FIELD OF THE INVENTION

The present invention relates generally to medical devices that facilitate the movement of biological fluids, transfer mechanical power to fluids, and/or derive power from moving fluids. The present invention employs a stacked disc impeller system in a variety of medical device applications involving the displacement of fluids including, for example, artificial hearts, and devices that move or handle blood, plasma and other biological fluids.

### BACKGROUND OF THE INVENTION

Various forms of impeller systems have been employed in a diversity of devices, including turbines, pumps, fans, compressors and homogenizers. The common link between these devices is the displacement of fluid, in either a gaseous or liquid state.

Impeller systems may be broadly categorized as having either a single rotor assembly, such as a water pump (U.S. Pat. No. 5,224,821) or homogenizer (U.S. Pat. No. 2,952,448); a single radially arranged multi-vaned assembly, such as a fan or blower (U.S. Pat. No. 5,372,499); or a multi-disc assembly mounted on a central shaft, as in a laminar flow fan (U.S. Pat. No. 5,192,183). Impeller systems employing vanes, blades, paddles, etc. operate by colliding with and pushing the fluid being displaced. This type of operation introduces shocks and vibrations to the fluid medium resulting in turbulence, which impedes the movement of the fluid and ultimately reduces the overall efficiency of the system. Use of a multi-disc impeller system overcomes this deficiency by imparting movement to the fluid medium in such a manner as to allow movement along natural lines of least resistance, thereby reducing turbulence.

U.S. Pat. No. 1,061,142 describes an apparatus for propelling or imparting energy to fluids comprising a runner set having a series of spaced discs fixed to a central shaft. The discs are centrally attached to the shaft which runs perpendicular to the discs. Each disc has a number of central openings, with solid portions in-between to form spokes, which radiate inwardly to the central hub through which the central shaft runs, providing the only means of support for the discs.

Similarly, U.S. Pat. No. 1,061,206 discloses the application of a runner set similar to that described above for use in a turbine or rotary engine. The runner set comprises a series of discs having central openings with spokes connecting the body of the disc to a central shaft. As in the aforementioned patent, the only means of support for the discs is the connection to the central shaft.

The designs of the disc and runner set of the aforementioned pump and turbine have significant shortcomings. For example, the discs have a central aperture with spokes radiating inwardly to a central hub, which is fixedly mounted to a perpendicular shaft. The only means of support for the discs are the spokes radiating to the central shaft. The disc design, the use of a centrally located shaft, and the means of connecting the discs to the central shaft create turbulence in the fluid medium, resulting in an inefficient transfer of energy. More specifically, as the discs are driven through a fluid medium, the spokes collide with the fluid causing turbulence, which is transmitted to the fluid in the form of heat and vibration. In addition, the centrally oriented shaft interferes with the fluid's natural path of flow, causing excessive turbulence and loss of efficiency. Furthermore, the spoke arrangement colliding with the fluid medium creates cavitations which, in turn, may cause pitting or other damage to the surfaces of components. Finally, the arrangement of the runner set does not sufficiently support the discs during operation, resulting in a less efficient system.

A variety of pumps are known and utilized within the field of artificial organs and other types of implanted devices. These pumps are, for example, utilized as mechanical implants for supporting or supplementing cardiac functions, or they may be utilized to take over an entire cardiac function, for example, replacing the function of one or both cardiac ventricles or for supporting the blood flow in a blood vessel. Some of these pumps are designed to support single ventricular function. Such pumps usually support the left ventricle, which pumps blood to the entire body except the lungs. Other pumps are used to provide biventricular function.

Mechanical blood pumps, such as centrifugal pumps and axial flow pumps, may be used, for example, for brief support during cardio-pulmonary operations, for short-term support while awaiting recovery of the heart from surgery, or as a bridge to keep a patient alive while awaiting heart transplantation. Centrifugal pumps direct blood into a chamber, typically against a spinning interior wall. A flow channel is provided so that the centrifugal force exerted on the blood generates flow.

Axial flow pumps use impeller blades mounted on a center axle, which is mounted inside a tubular conduit. Another type of axial flow pump, called the “Haemopump,” employs a screw-type impeller. Instead of using several relatively small vanes, the Haemopump screw-type impeller contains a single elongated helix, comparable to an auger used for drilling or digging holes. In screw-type axial pumps, the screw spins at very high speed (up to about 10,000 rpm). The entire Haemopump unit is usually less than a centimeter in diameter. The pump can be passed through a peripheral artery into the aorta, through the aortic valve, and into the left ventricle. It is powered by an external motor and drive unit.

The gaps between the outer edges of the blades and the walls of the flow conduit in axial rotary flow pumps produce turbulence and shear stresses. Red blood cells are particularly susceptible to shear stress damage as their cell membranes do not include a reinforcing cytoskeleton to maintain cell shape. The resulting lysis of red blood cells can result in release of cell contents which trigger subsequent platelet aggregation. Lysis of white blood cells and platelets may also occur in high shear stress environments. Sublytic shear stress is also undesirable because it leads to cellular alterations and direct activation and aggregation of platelets and white blood cells.

Another category of mechanical blood pumps is pulsatile pumps—the diaphragm type pump being the most common. Diaphragm pumps provide desirable pulsative flow and are reliable owing to their simplicity. Diaphragm pumps known in the art comprise a housing and a flexible, but not extensible, diaphragm that divides the interior of the housing into two chambers, namely a pumping chamber and a driving chamber. Diaphragms are conventionally fabricated from polyurethane, a flexible but not elastic material. The pumping chamber portion of the housing has an inlet and an outlet, each of which is equipped with a one-way flow check valve. The diaphragm is driven into and out of the pumping chamber mechanically, pneumatically or hydraulically. Mechanical drives typically include a pusher plate on the drive side of the diaphragm connected to a cam, solenoid or other device to impart reciprocal motion to the pusher plate and diaphragm. Alternatively, a drive fluid, either liquid or gas, may be used to reciprocally drive the diaphragm into and out of the pumping chamber.

One of the problems associated with diaphragm pumps is the formation of blood clots in the pump. The interior surfaces of the diaphragm and housing walls defining the pumping chamber are typically designed to have a very smooth surface in an effort to retard clotting. However, other attempts to reduce clotting have involved provision of a rough texture on the interior surfaces of the pumping chamber to encourage endothelial cells, which normally line the heart and blood vessels, to grow over the surfaces eventually providing a smooth surface. Both of these methods work to some degree, but formation of blood clots in the device remains problematic.

Bio-incompatibility is an issue with existing pump designs. Improper charges occurring on the inside of the pump surfaces can cause rapid accumulation of platelets, which can result in the clogging within the pump.

U.S. Pat. No. 5,693,091 discloses a surgically implantable reciprocating pump employing a check valve as the piston, which is driven by a permanent magnet linear electric motor, to assist either side of the natural heart. The pump is implanted in the aorta or pulmonary artery using vascular attachment cuffs such as flexible cuffs for suturing at each end with the pump output directly in line with the artery. The pump is powered by surgically implanted rechargeable batteries. In another embodiment of the implantable reciprocating pump, pairs of pumps are provided to replace or assist the natural heart or to provide temporary blood flow throughout the body, for example, during operations to correct problems with the natural heart.

U.S. Pat. No. 6,579,223 discloses a pump designed for pumping blood, comprising a bladder, the interior surface area and volume of which are changeable, i.e., it stretches and expands during the filling phase and elastically contracts to its normal relaxed size during the ejection phase. The bladder has a fluid inlet and a fluid outlet. A device, such as a vacuum pump, alternately expands and contracts the interior surface area and volume of the bladder. Most of the interior surface area of the bladder expands and contracts in each cycle. One or more check valves, or other means for causing substantially one-way fluid flow through the bladder, are also provided. The design of the pump decreases the likelihood of blood clots forming in the pump, decreases the risk of damage to blood cells, improves the pumping characteristics of the device, and decreases or eliminates the chance of foreign fluids passing into the blood stream should a tear or break occur in the bladder.

U.S. Patent Publication No. 2004/0024285A1 discloses a blood pump having a pump housing and an impeller disposed in the housing. The pump is driven by an electric drive, which includes at least two connection devices directly disposed at the housing for connection to an artery outside the heart. A pump conduit can be implanted in the aorta ascendens for relieving the left ventricle, and in the truncus pulmonalis or the pulmonalis furcation for relieving the right cardiac ventricle and other blood vessels to improve the blood circulation or elevate the pressure in a certain vascular section.

There is a need in the art for a more efficient means of displacing fluids, particularly biological fluids, without introducing unnecessary turbulence to the fluid medium and without damaging important components of the fluid medium.

### SUMMARY OF THE INVENTION

The present invention provides systems and methods for facilitating the movement of biological fluids and transferring mechanical power to biological fluids, as well as deriving power from biological fluids. Embodiments of the present invention exploit the natural physical properties of fluids to create a more efficient means of driving fluids, as well as transferring power from propelled fluids. In certain embodiments, the present invention provides pump assemblies, including dual chambered pump assemblies, for moving biological fluids such as, but not limited to, blood. The inventive pump assemblies incorporate at least one stacked disc impeller assembly. Stacked disc impeller systems suitable for use in the methods and systems of the present invention are described in U.S. Pat. Nos. 6,375,412 and 6,799,964, and U.S. Published Patent Application 2005/0019154 A1, which are incorporated herein by reference in their entireties.

According to one aspect of the present invention, an impeller assembly is provided that comprises a plurality of substantially flat discs and a plurality of connecting elements. The plurality of discs and optionally, spacing elements, are alternately arranged in a parallel fashion along a central rotational axis and held in tight association by connecting elements forming a stacked array. One or more first support plates may be fixedly connected to, or integral with, a central hub. One or more second support plates may be connectible to an opposing end of the stacked array of discs, thereby providing structural integrity to the impeller assembly.

Each disc comprises a viscous drag surface area having a central aperture. The viscous drag surface area is essentially flat, substantially smooth and preferably devoid of any substantial projections, grooves, vanes and the like. Discs of the present invention may further comprise one or more support structures, such as a series of support islets or other support structures, located on or in close proximity to the inside perimeter of the disc for receiving spacing and/or connecting elements. The discs may be interconnected by conventional structural elements, such as spacers and/or connecting rods attached to an interior perimeter portion of each disc and supporting plate. The connecting rods in turn are attached to a supporting structure, such as a central hub. A mechanism for rotating the impeller assembly, such as a motor or another drive mechanism, may drive the stacked disc array through the central hub or another supporting structure. In alternative embodiments, a central hub or other disc supporting structure may be connected to any conventional rotational energy translating mechanism, such as a drive shaft or the like.

In accordance with further aspects of the present invention, the parallel arrangement of the discs' central apertures in the stacked array generally define a central cavity of the impeller assembly, creating a fluid conduit. In addition, the plurality of stacked and generally aligned discs, with spacing elements and/or connecting elements maintaining the discs in relationship to one another, define a plurality of inter-disc spaces which are continuous with the central cavity of the stacked array. Fluid may flow freely between the plurality of inter-disc spaces and the central cavity of the stacked array. Pump systems of the present invention further comprise a mechanism for rotating the impeller assembly such that the plurality of discs are rotationally driven through a fluid medium, displacing and accelerating the fluid to impart tangential and centrifugal forces to the fluid with continuously increasing velocity along a spiral path, causing the fluid to be discharged from an outlet. The principle of operation is based on the inherent physical properties of adhesion and viscosity of the fluid medium which, when propelled, allow the fluid to adjust to natural streaming patterns and to adjust its velocity and direction without the excessive shearing and turbulence associated with traditional vane-type rotors or impellers.

According to further aspects of the present invention, the flow rate is generally in proportion to the dimensions and rotational speed of the discs. As the surface area of the discs is increased, the viscous drag surface area increases, as does the amount of fluid in intimate contact with the discs, producing an increased flow rate. As the number of discs is increased, the overall viscous drag surface area increases, which also results in an increased flow rate. In addition, as the rotational speed of the impeller assembly is increased, the tangential and centripetal forces being applied to the fluid increase, which will naturally increase the flow rate of the fluid.

Impeller assemblies and pumps incorporating impeller assemblies of the present invention have significant advantages over prior art pumps and impeller systems. The stacked-disc impeller assembly possesses significantly more fluid contact surface area in comparison to single rotor or vane designs, and thus operates at higher capacities and more efficiently. Elimination of the central shaft and creation of a central cavity within the impeller assembly contributes to efficiency and improved output, in addition to reducing friction and fluid turbulence.

Methods and systems of the present invention generate little heat during operation, thereby minimizing heating of the fluid medium. Pumps and/or circulating systems incorporating impeller assemblies of the present invention are especially useful for displacing temperature and turbulence sensitive fluids, such as biological fluids. The impeller systems of the present invention produce substantially no aeration or cavitation, even at high flow rates and high rotational speeds, and thus provide substantial safety and performance benefits in these applications compared to conventional pump systems. Impeller assemblies of the present invention may be incorporated into medical devices and apparatus involving the movement of fluids, such as devices for moving biological fluids, medicines, therapeutics, pharmaceutical preparations, and the like. Examples of such devices include heart pumps, circulatory pumps and fluid movement assist devices of all sorts, such as pumps in heart and lung bypass apparatus, pumps in filtration devices for use either inside or outside the body including artificial kidneys and livers, dialysis and plasmaphoresis devices, as well as injection pumps for the delivery of medicines, therapeutics, pharmaceutical preparations and the like. For use in pumping biological fluids, the inventive pump systems may be provided with connecters for connecting the pump system to one or more body lumens of a patient.

In one aspect, a stacked disc impeller assembly is incorporated as part of a blood pumping system, such as a heart pump. The inventive heart pump system promotes blood flow while producing very little turbulence, thereby minimizing damage to red blood cells and reducing platelet accumulation. It may be modularly constructed in a variety of different embodiments and the impeller assemblies may be rotated or otherwise adjusted to accommodate variations in vascular physiology of the recipient. The heart pump is provided with appropriate biocompatible connection(s) for installing in a recipient's cardiovascular system. For example, conduits may be provided for connecting the heart pump in situ to one or more of the inferior and superior vena cava, the pulmonary trunk, the pulmonary artery and the aorta.

In one embodiment, a heart pump system is provided with two stacked disc impeller assemblies, as described above, having a central drive assembly for driving both impeller assemblies. The drive assembly may comprise a magnetic drive system that interacts, for example, with one or more driving magnets and/or magnetic hubs associated with each disc stack. In this embodiment, the pump system is not directionally sensitive and operation of the pump in either direction of rotation may be controlled by operation of the magnetic drive system.

The housing of the heart pump system, which is constructed from a material that is biocompatible and encloses and provides complimentary surfaces for the impeller assemblies, is designed to fit in the cardiac cavity and to provide appropriate biocompatible connections to the subject's vasculature. The housing is substantially rigid and forms an interior chamber of sufficient volume to accommodate the impeller assemblies. The interior surface of the housing is generally smooth to avoid fluid discontinuities, with the housing being designed to accommodate the impeller and drive systems, and to provide substantially constant fluid flow in the interior chamber.

In another aspect, the present invention provides devices for the filtration of biological fluids in which the flow of biological fluid is assisted by a pump system including at least one stacked disc impeller assembly. Examples of such devices include, but are not limited to, artificial organs for the elimination of waste products from the body, such as artificial kidneys and livers. In such devices, which may be implanted in the body or may be used ex vivo, the pump assembly increases pressure thereby improving movement of toxic waste from the blood through molecular membranes for collection and removal without subjecting the blood components to excessive trauma. Use of the pump system thus reduces the strain of circulation in the filter mechanism located within the filtration device. In one such embodiment, the present invention provides a filtration device for filtering biological fluids, comprising: a housing; a fluid inlet connected to a first body lumen of a patient; (c) at least one filtering element formed from a semi-permeable membrane for receiving the biological fluid after it has entered the housing through the fluid inlet; (d) a stacked disc impeller assembly for applying pressure to the biological fluid; and (e) a motor for driving the impeller assembly; and (f) a fluid outlet connected to a second body lumen of the patient through which the biological fluid exits the housing after passing through the filtering element, whereby unwanted material is removed from the biological fluid as it passes through the filtering element.

In one embodiment, implantable devices incorporating the inventive pump assemblies, such as heart pumps, are conditioned prior to placement in a patient by culturing cells collected from the patient, or cells compatible with the patient, on inactive surfaces of the device. For devices employed for pumping blood, vascular cells are preferred and may be cultured on a compatible anchoring surface provided on the interior surfaces of the device and/or pump housing and/or on the interior of connective passages. Anchoring surfaces may be provided by polymeric materials, such as Dacron® or other bio-compatible fabrics, that are associated with the interior surface of the pump housing. Promoting the formation of a vascular cell layer that is compatible with the patient improves the biocompatibility of the device and may help to reduce platelet accumulation on device/pump surfaces.

As blood contains significant amounts of iron, the flow of blood through a device creates an electrical charge, which in turn leads to incompatibility of the device with a recipient's vessels. Accordingly, in another embodiment, a device/pump housing is provided with an interior surface having a polarity and charge distribution that approximates the polarity and charge distribution of the interior surface of a blood vessel wall, thereby improving the biocompatibility of the device and reducing platelet accumulation in the pump, which may otherwise produce clogging and malfunction of the pump. Preferably, the interior surface of the pump has a charge that matches the velocity charge of blood flowing through the pump. Materials such as urethane and other polymeric materials, such as polycarbonates, are suitable for the interior pump surface.

In yet a further embodiment, the present invention provides methods for pre-conditioning an implantable device prior to placement in, or attachment to, the body of a patient, such methods comprising providing an implantable device having an interior lined with a bio-compatible material that provides an anchoring surface for cells, such as Dacron®, and circulating a solution containing the cells, and optionally nutrients, through the housing for a period of time sufficient to deposit the cells on the lining. Preferably, the implantable device is subjected to an electrical charge of between about 0.1 to 10 V, either intermittently or continually, during at least a portion of the period of time that the housing is cultured with the cells, in order to improve migration of cells into the bio-compatible anchoring material. The cells may be autologous cells or, alternatively, may be compatible heterologous cells.

A heart pump system of the present invention is described in detail below. It will be understood that this is just one exemplary system and that the present invention encompasses many other methods and systems for displacing biological fluids incorporating a stacked disc impeller.

### BRIEF DESCRIPTION OF THE FIGURES

The present invention will be described in greater detail in the following detailed description, with reference to the accompanying drawings, wherein:

FIG. 1A illustrates a side view of a stacked disc impeller assembly suitable for use in medical devices of the present invention;

FIG. 1B illustrates a top view of an impeller assembly within a pump housing, with the cover removed exposing the inlet-side backing plate;

FIG. 1C depicts a side perspective of one type of pump housing;

FIG. 1D shows a top view of a pump cover with an inlet port;

FIG. 1E illustrates a side perspective of a pump cover;

FIGS. 2A and 2B show various embodiments of a support frame, wherein FIG. 2A shows a support frame for four rods and FIG. 2B shows a support frame for three rods and a center shaft;

FIG. 3 illustrates a cross-sectional schematic view of an embodiment of the inventive heart pump system;

FIG. 4 illustrates an exploded cross-sectional schematic view of the heart pump system of FIG. 3;

FIG. 5A is a side view of the exterior of an artery side housing section of the inventive heart pump;

FIG. 5B is a cross-sectional view of the motor side cavity of the artery side housing section of FIG. 5A;

FIG. 6A is a side view of the exterior of a vein side housing section of the inventive heart pump;

FIG. 6B is a cross-sectional view of the motor side cavity of the vein side housing section of FIG. 6A.

FIG. 7 is a cross-sectional view of an implantable filtration device of the present invention.

### DETAILED DESCRIPTION OF THE INVENTION

The present invention provides a pump system, such as a heart pump system, that allows fluid such as blood to flow with very little turbulence, thereby minimizing damage to cells such as red blood cells. This reduction in red blood cell damage can improve the survivability of recipients whose hearts are connected to the inventive heart pump system. The low turbulence generated by the inventive heart pump system also reduces the occurrence of improper charges on the interior surface of the pump system, thereby reducing the accumulation of platelets and minimizing the clogging problems often seen with known heart pumps. The inventive heart pump system has the additional advantages of modular construction, low fluid impact, low power consumption, and low noise. The heart pump may be employed as a single side assistive pump (vena cava side or aortic side) or as a complete replacement for support until a donor heart can be found. Since the system is not directionally sensitive, it can operate in either direction. The inventive heart pump system is ideal for pumping at low pressures, including the average pressure for the circulatory system of 100 mm (approximately 1.5 psi) @ 5 liters per minute.

In a preferred embodiment, the inventive heart pump system is provided with two impeller assemblies and a central driving motor for driving both impeller assemblies. The impeller assemblies may be rotated to accommodate variations in vascular position in the recipient. All surfaces of the impeller are active in pumping, thereby preventing stagnation.

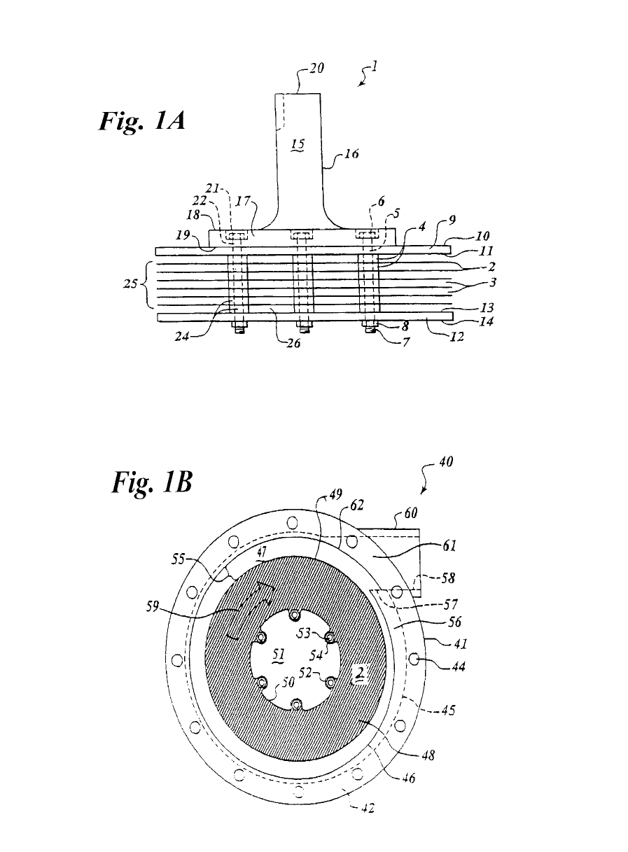

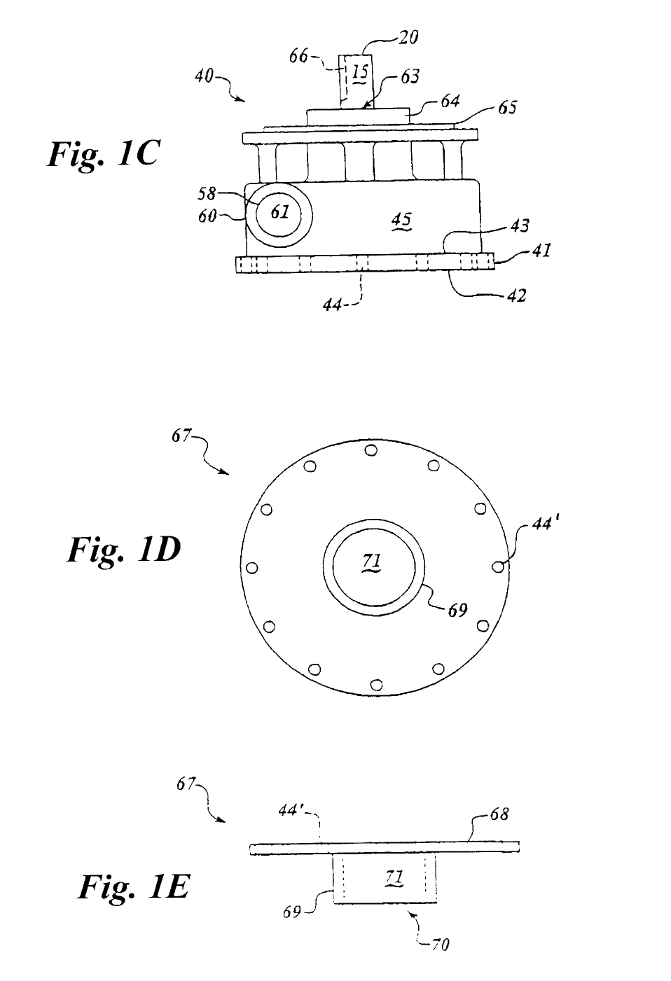

An impeller assembly of the present invention is illustrated in FIG. 1A. As shown in FIG. 1A, impeller assembly 1 comprises a plurality of viscous drag discs 2 arranged parallel to one another with distinct spaces 3 located between each disc. As shown in FIG. 1B, discs 2 are substantially flat with a central aperture 51, which defines an inside perimeter 50 of each disc 2. Face 48 of disc 2 forms the viscous drag surface area and defines the outer perimeter 49. The viscous drag surface area of disc 2 is essentially flat and devoid of any purposefully raised protrusions, engraved texturing, grooves and/or vanes. However, the surface area need not be completely devoid of any texture, and in certain applications may possess a roughened surface to provide additional friction for displacing fluid, provided the roughened surface does not create substantial disruptive turbulence in the fluid medium.

Along inner perimeter 50 of disc 2, a series of support structures is provided, such as support islets 52 protruding into central aperture 51. Alternative embodiments may comprise support structures that do not protrude into central aperture 51 and may include embodiments having support structures inset along or in close proximity to inner perimeter 50 of disc 2. Each support islet 52 contains a central aperture 53 which has been undercut 54. Alternative embodiments may comprise support structures, such as support islets 52, that are not undercut and may be essentially flush with, or projecting above, inner perimeter 50 of disc 2. The number of support islets 52 varies depending on the specific application. As described below, support islets 52 serve to interconnect and support a plurality of discs 2 to form a stacked array 25 of impeller assembly 1. Alternative types of support structures accommodating connecting structures may be employed to interconnect an array of discs 2 arranged in a stack. A preferred number of support structures may range from 3 to greater than 6. In the embodiment shown in FIG. 1B, 6 are shown. However, impeller assemblies comprising 3, 4 or 5 support structures are also contemplated by the present invention.

Discs 2 may be composed of any suitable material possessing sufficient mechanical strength and rigidity, as well as physical and/or chemical inertness to the fluid medium being displaced such as, but not limited to, resistance to extreme temperatures, pH, biocompatibility to biological fluids, and the like. Discs 2 may, for example, be composed of metal, metal alloys, ceramics, plastics, or the like. Optionally, discs 2 may be composed of a high-friction material to provide additional surface friction for displacing fluid. In general, the dimensions of disc 2, such as overall perimeter, central aperture diameter and width, are variable and determined by the particular use. The size of the housing and the desired flow rate of a particular fluid also influence the size and number of discs 2 in the impeller assembly. Because only the viscous drag surface areas of the discs 2 significantly affect the flow of fluid, it is desirable that the discs 2 of the impeller assembly 1 be as thin as the specific application will allow. Therefore, it is preferable that discs 2 have a thickness capable of maintaining sufficient mechanical strength and rigidity against stresses, pressures and centrifugal forces generated within the pump, yet are as thin as conditions allow to reduce unnecessary turbulence. Discs 2 may be from 1/1000 to several inches in width, depending on the application. The materials and dimensions of the discs 2 are largely dependent on the specific application involved, in particular the viscosity of the fluid, the desired flow rate and the resultant operating pressures. Similarly, the number of discs 2 in impeller assembly 1 may vary depending upon the particular use. In some embodiments, impeller assembly 1 comprises between 4 and 100 discs, in preferred embodiments between 4 and 50 discs, and in yet additional embodiments between 4 and 25 discs. In general, for a specific volume and pressure of fluid, the larger the diameter of the discs employed in the impeller assembly, the smaller the number of discs that are required, and vice versa.

In certain embodiments, particularly small applications such as small pumps, the entire impeller assembly 1 may be made of plastics or other material that may be formed by any conventional methods, such as injection molding or other comparable methods, to form an integrated impeller assembly 1 rather than the individual components described below. Alternatively, embodiments of impeller assembly 1 may be formed of rigid plastics, ceramics, reinforced materials, die cast metals, machined metal and/or metal alloys or powdered metal assemblies for applications requiring greater mechanical strength.

Although outer and inner perimeters of discs 2 having circular forms and circular configurations are generally preferred, alternative configurations may be used. For example, curved profiles may be employed along the inner periphery between support structures 52. Such curved profiles are preferably radially symmetrical and do not produce turbulence during operation. The stacked discs 2 forming an impeller assembly 1 preferably have the same configuration and are aligned in a consistent fashion to form the array.

The inter-disc spaces 3 between discs 2 may be maintained by a plurality of spacers 4, which, together with the discs 2, create a stacked array 25 of alternating discs 2 and spacers 4. In one embodiment, spacers 4 possess a central aperture 24 complementary with the islet aperture 53 of support islets 52. Spacers 4 may be of any suitable conformation that does not create undue turbulence in the fluid medium, such as round, oval, polygonal, oblong and the like, and composed of any suitable material compatible with other components of the pump system and the fluid being displaced, such as metals, metal alloys, ceramics and/or plastics. Spacers 4 may have a uniform or non-uniform area throughout their cross-section and their profile may present straight lines or curved lines.

Alternative embodiments of the present invention may have spacers 4 integrated into discs 2 or connecting structures rather than distinct components such as, but not limited to, one or more raised sections integrated with islets 52 of inner rim 50. The dimensions of spacers 4 are additional variables in the design of the impeller system and are dependent on the specific applications. For example, the inter-disc spacing, and therefore the height of spacers 4, may be from 1/100 to greater than 2 inches, preferably from 1/32 to 1 inch, and more preferably from 1/16 to ½ inch. In general, the spacing of discs 2 should be such that the entire mass of fluid is accelerated to a nearly uniform velocity, essentially equivalent to the velocity achieved at the periphery of the discs 2, thereby generating sufficient pressure by the combined centrifugal and tangential forces imparted to the fluid to effectively and efficiently drive the fluid. The greater the height of spacers 4, the greater the inter-disc space 3, which has a direct effect on the negative pressure generated within the pump housing.

In the embodiment illustrated in FIG. 1A, impeller assembly 1 further comprises a central hub 15. Central hub 15 serves to transfer rotational power applied to the receiving end 20 of the shaft section 16 to the stacked array 25 of discs 2. Central hub 15 possesses a flange section 17 distal to the shaft section 16, having an inside face 19 and outside face 18. Inside face 19 of flange section 17 may contact an outside face 10 of a first reinforcing backing plate 9. Alternative embodiments of the present invention also encompass designs wherein central hub 15 and first reinforcing backing plate 9 are one integral work-piece, whether cast or machined. Inside face 11 of first reinforcing backing plate 9 preferably contacts a plurality of spacers 4. A second reinforcing backing plate 12, is located distal to the stacked array 25 of spacers 4 and discs 2. In a preferred embodiment, first and second reinforcing backing plates 9 and 12 have substantially the same design and diameter as viscous drag disc 2 shown in FIG. 1B.

As shown in FIG. 1A, first and second reinforcing backing plates 9 and 12 of impeller system 1 are preferably thicker than the discs 2, thereby providing additional mechanical support to the stacked array 25 of discs 2 to counteract the negative pressure created in the inter-disc spaces, particularly at the outside periphery of the discs 2. The reinforcing backing plates 9, 12 support the discs 2 by providing a solid and relatively inflexible surface for the discs 2 to pull against, thereby reducing the tendency of the discs 2 to flex and deflect inwardly in the inter-disc spaces. The thickness of the reinforcing backing plates 9, 12 is largely dependent on the diameter, and therefore the surface area, of the discs 2. As a general principle, the reinforcing backing plates 9, 12 may be approximately four times as thick as the discs 2, but this relationship may vary depending on the particular application.

Central hub 15, first reinforcing backing plate 9, stacked array 25 of spacers 4 and discs 2, and second reinforcing backing plate 12 are interconnected by a plurality of connecting structures 5, such as connecting rods. In one embodiment, connecting rods 5 pass through apertures 22 of flange section 17 of central hub 15, and through the complementary apertures of first reinforcing backing plate 9, spacers 4, discs 2 and second reinforcing backing plate 12. Distal ends 7 of connecting rods 5 are secured against the outside face of second reinforcing backing plate 12 by any suitable retaining means 8. Proximal ends 6 of connecting rods 5 have a securing means that is seated in countersunk opening 21 of apertures 22 of flange section 17. Alternative embodiments may not require a countersunk configuration and may include any operable configuration of the elements described herein. It will be recognized that although the connecting structures are illustrated in the form of rods, other connecting structures may also be used. The connecting structures may have a uniform or non-uniform cross-sectional area over their length, and they may have a straight line or curved profile. Spacers 4 may be mountable on or integrated with the connecting structures. The primary function of the connecting structures is to maintain the discs 2 forming the array 25 in fixed relationship to one another.

Retaining device 8, such as a conventional nut threaded onto the distal end 7 of the connecting rod 5, or any other suitable retaining device, is secured to draw second reinforcing backing plate 12 towards proximal end 6 of connecting rod 5, thereby drawing all components into tight association. Although the embodiment illustrated herein shows a through-bolt arrangement for connecting the sub-components of the impeller assembly 1, the present invention also anticipates the use of other similar connecting means, such as a stud-bolt arrangement for the connecting rods, having a threaded proximal and distal end, and a welded-stud arrangement, where the connecting rods 5 are secured to the central hub 15 and the second reinforcing backing plate 12 by welded, soldered or brazed connections.

In some embodiments of impeller assembly 1, a support frame 80 may be provided at one end of the rods 5 to secure the rods 5. Where a central hub 15 is included on one end of the array 25 of stacked discs 2, the support frame 80 may be secured to the opposite end of the array 25. The support frame 80 may be of various shapes and sizes in order to inhibit movement of the rods 5. If a support frame 80 is not employed, the high fluid pressure may cause a non-secured end of the rods 5 to shake or otherwise move its position. As a result, the spaces between the discs 2 may vary with movement of rod 5, affecting fluid flow. The support frame 80 may thus be employed to provide more uniform and constant spacing between the discs 2.

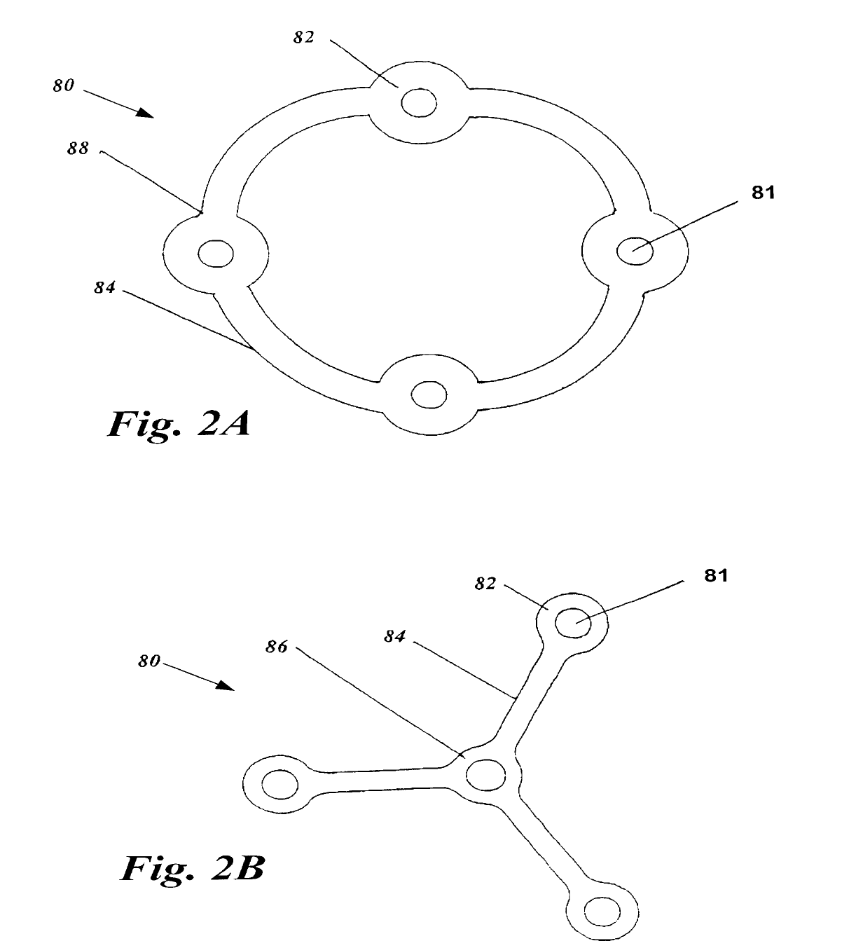

Two different embodiments of support frame 80 are illustrated in FIGS. 2A and 2B. The support frame 80 includes a plurality of rod attachments 82, wherein each rod attachment 82 holds one of the rods 5. FIG. 2A shows a support frame 80 having four rod attachments 82 for supporting four rods. However, any number of rod attachments 82 may be included, depending on the number of rods 5 provided. Various types of rod attachments 82 may be employed to inhibit movement of the rods 5, such as an opening 81 through which a rod end, such as the distal end 7 of a connecting rod 5, is extended. The opening 81 may permit the retaining device to draw the support frame 80 towards proximal end 6 of connecting rod 5, thereby drawing all components into tight association, rather than, or in addition to, securing a second reinforcing backing plate 12, as described above. The support frame 80 may also include arms 84 coupled to the rods 5 that connect to the rods 5 in various patterns, such as a web, circle, square, triangular, etc. At least one arm end 88 is coupled to at least one rod attachment 82, and at times each arm end is coupled to a different rod attachment 82. In embodiments that include a central shaft, the support frame 80 may also include a shaft attachment 86 as depicted in FIG. 2B. The shaft attachment 86 may be connected to the rod attachments by arms 84 that each may extend from the shaft attachment 86 at a first arm end 88 and to each of the rod attachments at the other, i.e. second, arm end 88. Preferably, the components of the support frame 80 are only as big as necessary to support the rods and/or shaft. For example, the rod attachments 82 and shaft attachments 86 are preferably slightly larger in diameter than the respective rods and shaft. In addition, the arms may also have a small diameter. This conservative size of the support frame 80 results in less disruption to fluid flow and therefore in less turbulence, and/or requires less material, than other designs that employ a supporting plate.

The use of support frame 80 is especially beneficial with embodiments of impeller assemblies that include a large array of stacked discs. The support frame 80 is also useful for applications where the discs 2 rotate very fast. The support frame 80 stabilizes the discs 2 thereby inhibiting any discs 2 from moving off center and/or flexing.

As shown in FIG. 1A, alignment of the central apertures of the two reinforcing backing plates 9, 12 and the stacked array 25 of discs 2 forms a central cavity 26 within the impeller assembly 1. Supporting the discs and backing plates at the inside perimeter eliminates the central shaft employed in previous designs, as well as the spokes used to attach the discs 2 to the central shaft, thereby eliminating the turbulence created by the central shaft and associated spokes. Where a shaft does not extend past the first backing plate 9 and into the central cavity 26, the central cavity 26 is devoid of a shaft. The central cavity 26 permits the fluid to flow in a more natural line into the impeller assembly 1 without the churning effect of the shaft and spokes employed in conventional pumps.

In one embodiment, the stacked impeller assembly disclosed herein may be employed ex vivo to facilitate the movement of biological fluids, such as blood, or to facilitate the administration of therapeutic and/or diagnostic compositions.

FIG. 1C illustrates a housing 40 of an inventive pump system that may be employed for ex vivo purposes. FIG. 1B illustrates the pump system with second reinforcing backing plate 12 removed to reveal the most distal disc 2 of the stacked array 25. Housing 40 is of any conventional design that provides a complimentary surface for the impeller assembly. The housing 40 comprises an outer wall 45 and inner wall 46 of the housing body, forming an interior chamber 47 of sufficient volume to accommodate the impeller assembly, yet maintain a gap 55 between the impeller assembly and the inside wall of the housing. The inner wall 46 provides a complementary surface for the impeller system to draw against, and gap 55 permits movement of the fluid within the housing 40 and creates a zone of high pressure. The volume area defined by the gap 55 affects flow rate and operating pressure. In certain embodiments, the total gap volume should be between 10 and 20% greater than the inlet volume area, but may be smaller or larger, depending on the application. Additional factors to be considered in determining the gap volume are output pressure, and the sheer mass, viscosity and particulate size of the fluid medium. The pump housing 40 further comprises a housing flange 41 with a series of holes 44 extending from the face plate 42 of the flange 41 through to the underside 43 of the flange 41. The inner wall of the housing forms a fluid catch 56 by an inwardly angling extension of the wall to create a shoulder 57, which is continuous with the inner wall 58 of an outlet port 60 having a central aperture 61. The inner wall of the housing 40 has an opening 62 to permit fluid to flow through the central aperture 61 of the outlet port 60. Alternative embodiments may utilize any conventional pump housing incorporating impeller assemblies of the present invention and not be limited to the exemplary embodiment presented herein.

The impeller assembly shown in FIG. 1A may be oriented within the internal chamber 47 of the housing 40 by threading the receiving end 20 of the central hub 15 through a centrally oriented opening 63 of the bearing/seal assembly 64 such that the shaft section 16 of the central hub 15 is securely held and supported by the bearing/seal assembly 64. Bearing/seal assembly 64 is integrated into the rear plate 65 of the pump housing 40 by conventional mechanisms. One possible configuration has the bearing/seal 64 as a cartridge unit (although the bearing and seals may be separate units) that is press-fit onto the shaft and then mounted in the housing 40. The bearing/seal assembly 64 may be of any conventional configuration that will provide sufficient support for the impeller assembly 1, permit as friction-free radial movement of the shaft as possible, and prevent any leaking of fluid from the internal chamber.

In this embodiment, the pump system may be driven by any drive system capable of imparting rotational movement to the shaft 16 of the central hub 15, thereby imparting rotational movement to the entire impeller assembly 1 within the internal cavity of the pump housing 40. The receiving end 20 of the central hub 15 may be of various configurations, such as keyed, flat, splined, and the like, to allow association with various motor systems. The exemplary embodiment shown in FIG. 1C has a standard shaft configuration, which has been keyed with a receiving notch 66 formed at the receiving end 20 of the shaft 16 for receiving a complementary retaining device associated with the drive system. Other examples include flex-joints, universal joints, flex-shafts, pulley systems, chain-drive, belt-drive, cog-belt-drive systems, direct-couple systems, and the like. Any drive system, such as a motor or comparable device, that directly or indirectly imparts radial movement to the impeller assembly 1 through the shaft 16 may be employed with the present invention. Suitable drive systems include motors of all types, including the magnetic drive system described above.

The inlet port cover 67, as shown in FIGS. 1D and 1E, has a circumference comparable to the circumference of housing flange 41, and has a series of apertures 44′ that are spatially oriented to be complementary to apertures 44 in housing flange 41. Inlet port cover 67 is attached to the pump housing 40 by securing inside face 68 of inlet port cover 67 to face plate 42 of housing flange 41 and is fixedly attached by any conventional securing devices through complementary apertures 44, 44′. In the context of the present invention, the term “fixedly” does not necessarily mean a permanent, non-detachable attachment or connection, but is meant to describe a variety of connections well known in the art that form tight, immovable junctions between components. In some embodiments, for example, fixed connections may be detachable. Face plate 42 of inlet port cover 67 defines the ceiling of internal chamber 47 of the pump housing. Fluid is drawn into opening 70 of inlet port 69 and through inlet port conduit 71 to internal chamber 47 of the housing.

Operationally, internal chamber 47 of the pump is primed with a fluid compatible to that being displaced. The drive system is activated to impart radial movement to shaft 16 of central hub 15, turning stacked array of discs 25 through the fluid medium in the direction of arrow 59. Impeller assemblies of the present invention operate in either direction of rotation. As discs 2 of the impeller assembly are driven through the fluid medium, the fluid in immediate contact with viscous drag face 48 of discs is also rotated due to the strong adhesion forces between the fluid and disc. The fluid is subjected to two forces, one acting tangentially in the direction of rotation, and the other centrifugally in an outward radial direction. The combined effects of these forces propels the fluid with continuously increasing velocity in a spiral path. The fluid increases in velocity as it moves through the relatively narrow inter-disc spaces 3 causing zones of negative pressure at the inter-disc spaces. The continued movement of the accelerating fluid from inside perimeter 50 of discs to outside perimeter 49 of discs further draws fluid from central cavity 26 of the impeller assembly, which is essentially continuous with inlet port conduit 71 of inlet port 69. The net negative pressure created within internal chamber 47 of the pump draws fluid from an outside source connected by any conventional means to the inlet port.

As fluid is accelerated through inter-disc spaces 3 to outside perimeter 49 of discs 2, the continued momentum drives the fluid against inner wall 46 of housing chamber 47 creating a zone of higher pressure defined by gap 55 between outside perimeter 49 of discs 2 and inner wall 46 of housing chamber 47. The fluid is driven from the zone of relative high pressure to a zone of ambient pressure defined by outlet port 60 and any further connections to the system. The fluid within the system may circulate a number of times before being displaced through the outlet port. Fluid catch 56 of inner wall 46 serves to impel the flow of circulating fluid into the central aperture of the outlet port.

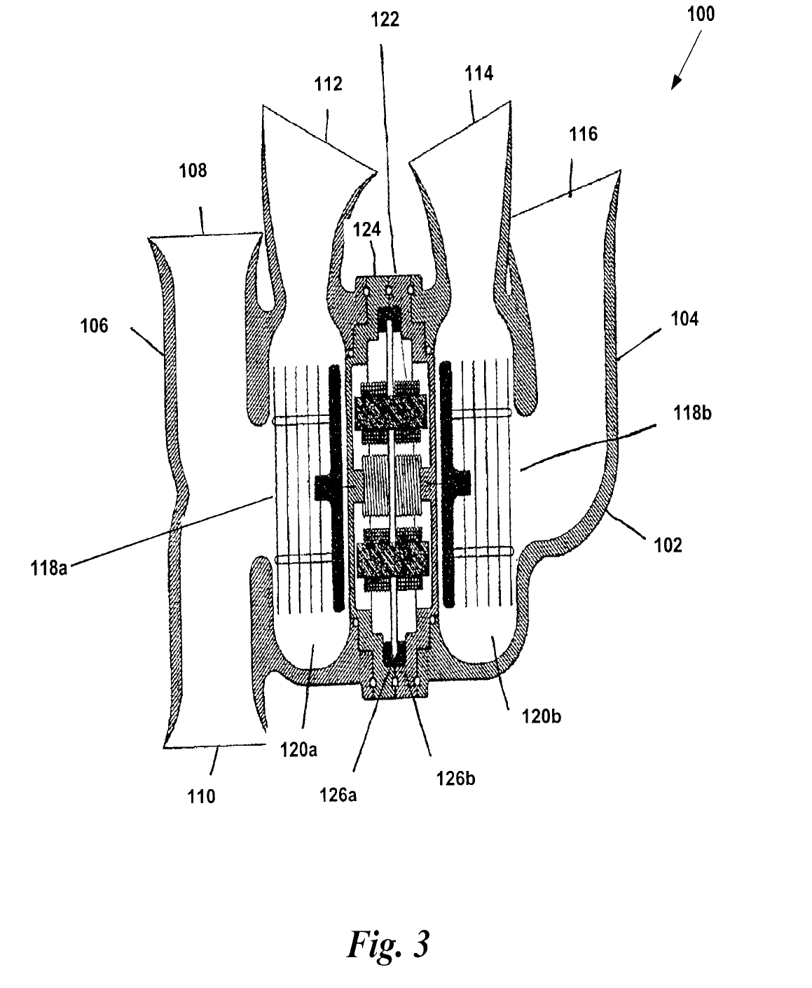

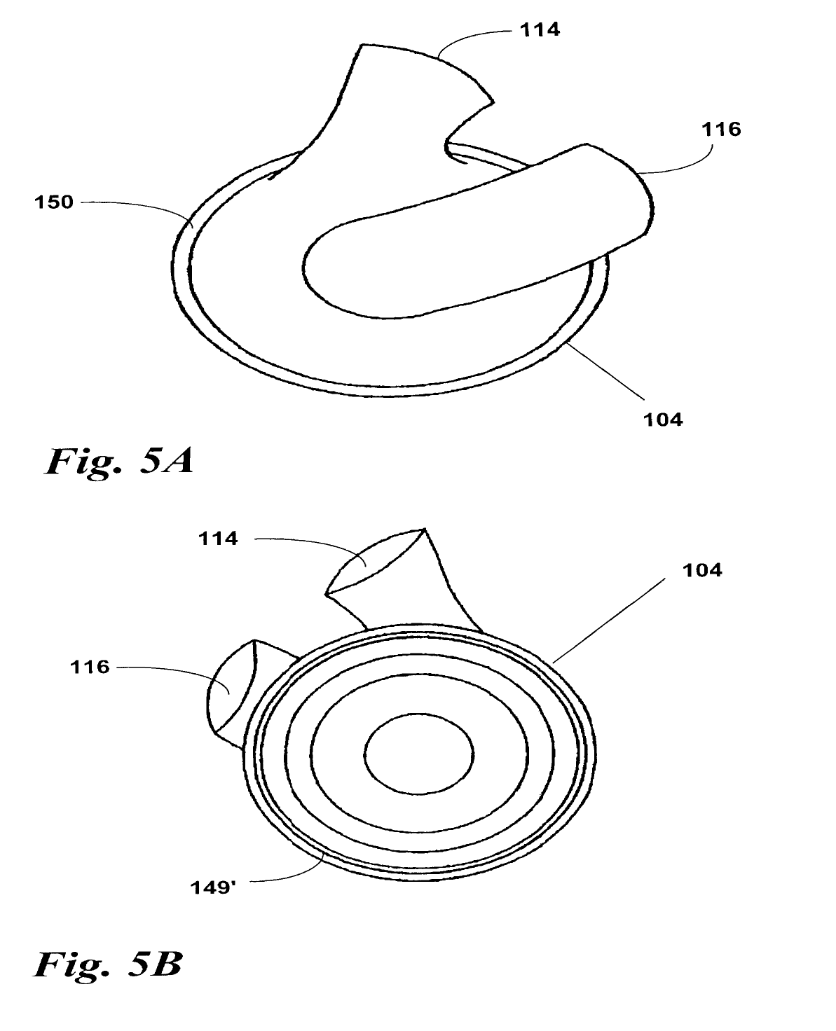

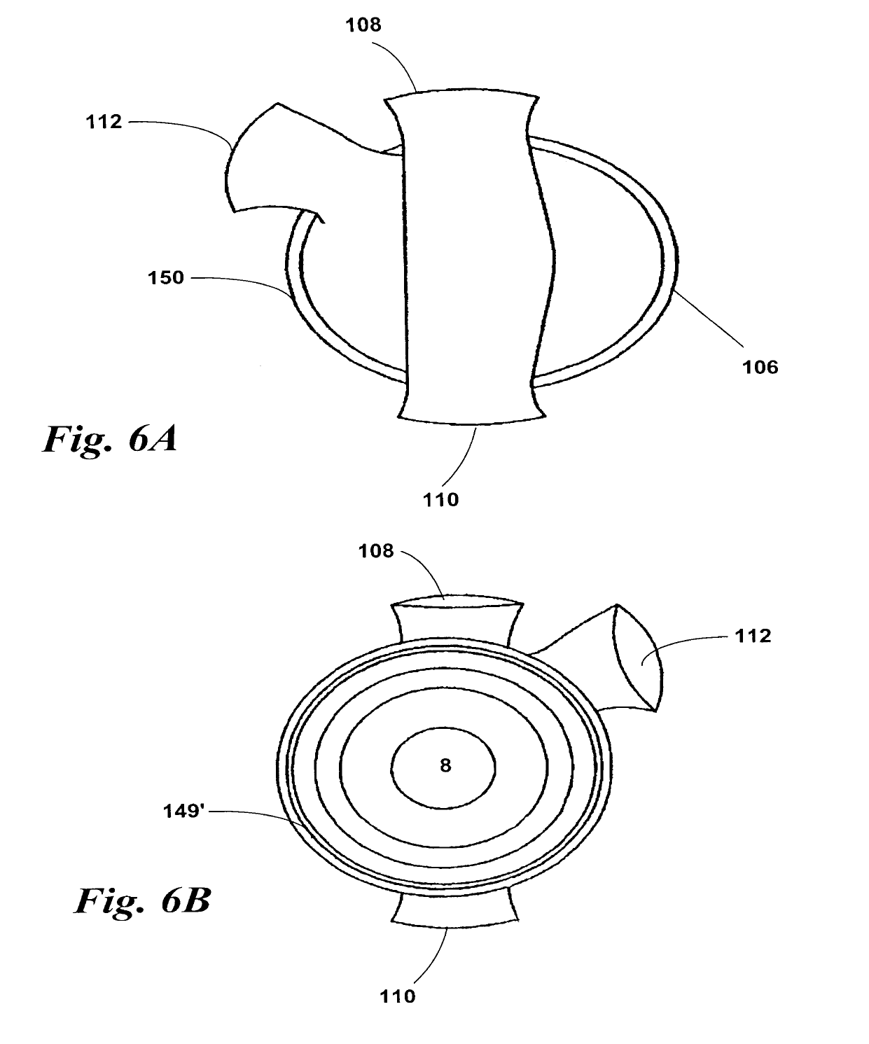

In an alternative embodiment, the inventive impeller systems are employed in a pump that may be employed either ex vivo or in vivo, such as in a heart pump system. FIG. 3 shows a heart pump system 100 of the present invention incorporating two impeller assemblies 118a and 118b. The impeller assemblies 118a and 118b are driven magnetically to eliminate sealing problems and may also be magnetically suspended. The heart pump system 100 includes a housing 102 composed of an artery side housing section 104 and a vein side housing section 106 (also illustrated in FIGS. 5A and B and FIGS. 6A and B), and is provided with several connections for connecting the heart pump system 100 to a recipient's cardiovascular system using methodology well known to those of skill in the art. Connection 108 connects to the superior vena cava, with connection 110 connecting to the inferior vena cava. Connection 112 connects to the pulmonary trunk (to the lungs); connection 114 connects to the aorta; and connection 116 connects to the pulmonary artery (from the lungs).

Impeller assemblies 118a and 118b are contained within interior chambers 120a and 120b, which are of sufficient volume to accommodate, and also provide complementary surfaces for, the impeller assemblies 118a and 118b. Motor assembly 122 is positioned within motor housing 124 which is formed of two halves, or sections, 126a and 126b.

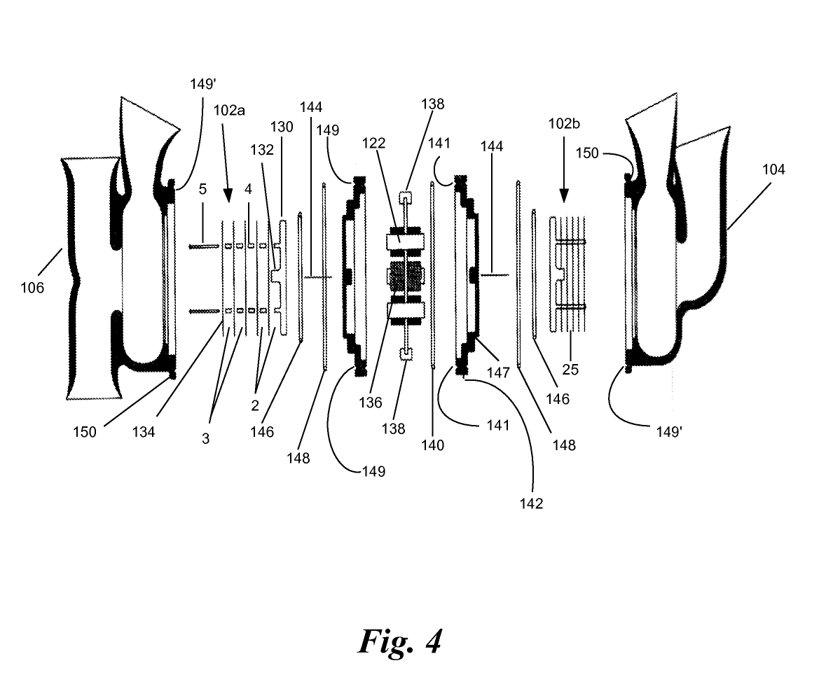

As shown in FIG. 4, each of the impeller assemblies 118a and 118b comprises a plurality of discs 2 arranged parallel to one another with distinct spaces 3 located between each disc 2 as described above. The inter-disc spaces 3 are maintained by a plurality of spacers 4 which, together with the discs 2, create a stacked array 25 of alternating discs 2 and spacers 4. The number of discs 2 in impeller assemblies 118a and 118b may vary.

Each of the impeller assemblies 118a and 118b further comprises a magnetic hub 130. Magnetic hub 130 may be provided with at least one passage 132 at, or near, its center that allows a small amount of fluid to be passed across the magnetic face to cool it and to provide active flow, thereby preventing stagnation zones. Magnetic hub 130 and stacked array 25 of discs 2 and spacers 4 are interconnected by a plurality of connecting structures, such as support pins or rods 5. The connecting rods 5 pass through the apertures 53 provided in discs 2 and apertures 24 provided in spacers 4, and are secured to magnetic hub 130. A driving magnet (not shown) may be placed (preferably cast) in disc 134 that is most distal from magnetic hub 130, with the remainder of the discs 2 in stack 25 being attached to magnet disc 134 by means of the connecting structures or pins 5.

Motor assembly 122, which is located between impeller assemblies 118a and 118b, preferably includes a four pole, five pole, or six pole type motor. Drive motor 136 of motor assembly 122 is preferably a “flat style”, similar in construction to a compact disc drive but considerably smaller and with a smaller wattage. The motor assembly 122 is supported in motor housing 124, by means, for example, of resilient support rings 138 which are connected to one half of the motor housing 152. The two halves, or sections, 126a and 126b of motor housing 124 are preferably sealed with an O-ring 140 which is received in grooves 141. At least one of motor housing sections 126a and 126b is provided with an electrical connector 142.

In the illustrated embodiment, impeller assemblies 118a and 118b rotate on support pins 144. However, one of skill in the art will appreciate that the impeller assemblies may alternatively rotate on bearings, preferably constructed of very hard materials such as sapphire.

Each of the artery side housing section 104 and the vein side housing section 106 of housing 102 is sealed by means of two O-rings 146 and 148. First O-ring 146 is located close to lip 147 of motor housing 124 in order to prevent blood from stagnating in gaps near the blood path. Second O-ring 148, which is received in grooves 149 and 149′ seals the exterior portion of housing 102.

The entire heart pump system 100 may be held together by means of a single clamp (not shown) placed around the outside of clamping surface, or lip, 150 provided on artery side housing section 104 and vein side housing section 106 in proximity to O-ring grooves 149′.

In a preferred embodiment, the housing 102 is constructed of cast polycarbonate plastic. The high differential charge between the polycarbonate plastic and vessel walls of the recipient can cause platelet accumulation. To reduce platelet accumulation, the pump housing 102 may be lined with a material such as Dacron® to provide an anchoring surface for a recipient's vascular cells on the interior surface 128 of the pump housing 102. Dacron® is a condensation polymer obtained from ethylene glycol and terephthalic acid. Its properties include high tensile strength, high resistance to stretching, both wet and dry, and good resistance both to degradation by chemical bleaches and to abrasion.

The Dacron® lining allows the formation of a vascular cell layer from the recipient's donated vascular tissue on the interior surface 128 of the pump housing 102. In addition to the interior surface 128 of the pump housing 102, Dacron® lining may also be applied to the interior surfaces of passages 108, 110, 112, 114 and 116 that connect with the recipient's cardiovascular system. This reduces problematic bio-incompatibility issues and platelet accumulation, by providing a near equal cellular charge level in the majority of the exposed surfaces that contact the blood. The exterior of the housing 102 and the motor housing 124 generally do not require the Dacron® lining. The Dacron® lining may be etched with a mild acid in order to improve cell attachment and reduce the total charge that might otherwise cause cellular rejection of any point in its surface.

Preferably, the pump housing 102 lined with Dacron® lining is cultured with the recipient's cells before the heart pump system 100 is connected to a recipient's cardiovascular system. Alternatively, the pump housing may be cultured with bio-compatible heterologous cells in place of autologous cells. The proper culturing of the housing 102 usually takes up to two to three weeks. Cells are grown in standard cell culture media circulating through the housing 102 and connective passages in a culture tank utilizing a pump to maintain proper circulation at very low pressures in the culturing system. This may be achieved using a culture tank system similar to those currently employed to culture skin grafts, except that the media is gently circulated throughout the housing to deposit cells on the Dacron® lining evenly. Preferably, an electrical charge of between 0.1 to 10 V, more preferably between 0.5 to 1.5V, is applied to pump housing 102 during culture. The tank is preferably small to reduce the operating volume and is also temperature, voltage and pH controlled to optimize growth.

If there is an urgent need for the heart pump system 100, it can be implanted without the Dacron® lining or deposition of cultured vascular cells. In this case, a conventional urethane lining may be used in place of the Dacron® lining. Heart pump systems that employ a conventional lining are preferably employed for a shorter time than those that have been cultured with the recipient's vascular cells, due to the problem of bio-incompatibility. Those of skill in the art will appreciate that the Dacron® lining and associated vascular cell culture described herein may be usefully employed with any device that is designed to be implanted within a recipient's body or to receive a biological fluid, such as blood.

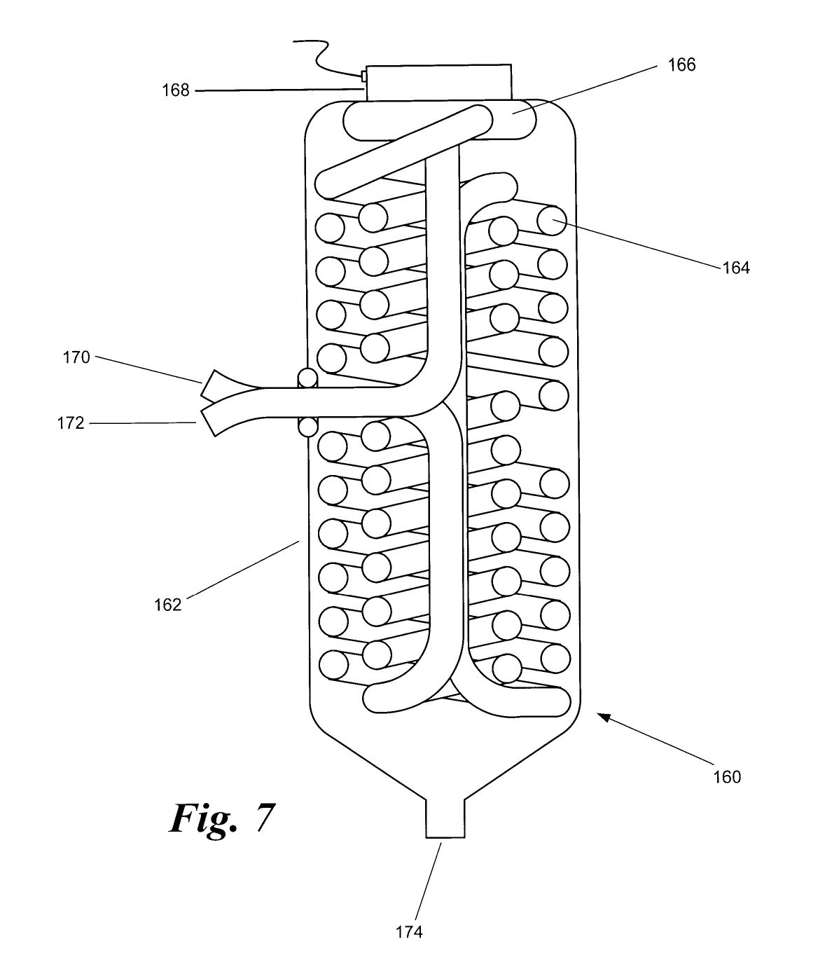

FIG. 7 shows an inventive device for filtering biological fluids for use, for example, as an artificial kidney. Filtration device 160 comprises a housing 162 constructed, for example, from a bio-compatible plastic that is durable and substantially rigid. Housing 162 contains a generally coiled length of tubing 164 formed from a semi-permeable membrane which through which toxins, but not blood cells, are able to pass. Semi-permeable membranes that may be effectively employed in the inventive filtration device are well known in the art and include those currently employed in standard dialysis techniques. While the semi-permeable membrane employed in the embodiment illustrated in FIG. 7 is in a generally tubular form, one of skill in the art will appreciate that the semi-permeable membrane may be provided in other shapes or forms, including flat, spiral and the like.

Pump 166, which is driven by drive motor 168, comprises at least one stacked impeller assembly as described in detail above. Fluid inlet 170 may be connected to an artery of a patient, with fluid outlet 172 being connected to a vein of the patient. During operation, blood enters filtration device 160 through fluid inlet 170 and passes through tubing 164 assisted by pump 160. The pressure provided by pump 160 forces toxic waste material from the blood through the semi-permeable membrane and the waste-depleted blood exits the device through fluid outlet 172. The toxic waste material exits the device through waste outlet 174 and may be passed to the bladder for elimination from the body or, alternatively, may be collected in a bag external to the body for disposal.

For use ex vivo, fluid may be continuously passed through housing 162, on the outside side of tubing 164 to aid in removal of toxic materials from the device.

### EXAMPLE 1

### Comparison of Viscous Drag Pump with Conventional Vane-Type Pump in Pumping Viscous Fluid

A direct comparison of a standard pump, which utilized a typical rotor assembly with vanes, was tested against the a pump of present invention. Two identical ⅛ horsepower 3650 rpm motors were fitted with different impeller assemblies. Pump A possessed a conventional vane-type rotor assembly, and pump B possessed the viscous drag impeller assembly disclosed herein. To determine the comparative efficiency of the two types of pumps, the amount of waste oil pumped over time was monitored. The standard pump was unable to transfer the waste oil and was found to severely overheat during the course of the trial. In contrast, the pump utilizing the viscous drag assembly was able to circulate the oil without strain on the motor.

To facilitate circulation of the viscous fluid and thereby compare the relative efficiency of the two pump designs, the waste oil was heated to 140° F. The pump equipped with the viscous drag assembly was able to transfer three gallons/minute in contrast to only one gallon/minute for the standard pump.

### EXAMPLE 2

### Comparison of Impeller Assembly with Standard Rotor

A controlled comparison of a standard rotor and an impeller assembly of the present invention was performed. Two 115 V, ½ hp pump motors (Dayton model # 3K380) were used in this study. One pump was fitted with a conventional rotor pump head (Grainger model #4RH42) having a 3.375″ diameter and a rotor depth of ⅜″, the other pump was fitted with an impeller assembly of the present invention having a 3.375″ diameter, but a 2″ rotor depth. All motors, bases, plumbing, valves and the like were identical. With valves shut and pumps running, both systems used 7.7 amps. Below is a comparison of the two systems.

| Comparison of Conventional | Standard | Impeller |
|---|---|---|
| Rotor to Impeller Assembly | Rotor | Assembly |
| Pressure: Valves shut | 17 psi | 19 psi |
| One Valve Open | 10 psi | 13 psi |
| Both Valves Open | — | 10 psi |
| Gallons per minute (+/−5%) | 24.6 | 30 |
| One Valve Open | | |
| Gallons per minute (+/−5%) | — | 48 |
| Both Valves Open | | |
| Amp Readings While Pumping | 8.9 amps | 10.3 amps |

Further analysis comparing a conventional rotor and an impeller assembly of the present invention having the same diameter and rotor depth resulted in similar volume output. Notably, an increase in impeller assembly depth from ⅜″ to 2″ resulted in only a 10% increase in power consumption, but a significant increase in volume output. Throughout the studies, the noise and vibration levels for the pump employing an impeller assembly of the present invention were significantly less than that of the pump fitted with a conventional rotor.

### EXAMPLE 3

### Comparison of Impeller Assembly Centrifugal Pump with Standard Centrifugal Pump having a Bladed Impeller

Several short-term and long-term tests comparing centrifugal pumps (0.5 HP and 1.5 HP) having an impeller assembly of the present invention with standard 0.5 and 1.5 HP centrifugal pumps having a bladed impeller were completed. The tests confirmed that conventional bladed impeller pumps suffer efficiency losses when operated at lower than 50% of maximum system pressure. For example, current consumption went flat when the conventional 1.5 HP centrifugal pump operated under 18 psi (50%). The conventional 1.5 HP centrifugal pump was not usable at pressures under 18 psi and wasted energy. The 0.5 HP centrifugal pump incorporating the impeller assembly of the present invention performed well, providing durability and silent operation. Even when operated at pressures of 2.45 psi, the output water was clear. The conventional bladed impeller pump produced aeration at 8 psi and was very loud. While testing the 1.5 HP pump incorporating the impeller assembly of the present invention, it was estimated to have diminished the noise level by at least 20 db compared to the conventional 1.5 HP bladed impeller pump. The centrifugal pumps incorporating the impeller assembly of the present invention were silent or nearly silent at all pump volumes and speeds.

Most fluid-moving pumps operate at an industry standard of 3450 rpm or slower. The centrifugal pump incorporating the impeller assembly of the present invention easily operates to pump fluid at 5500 rpm. When operating to move gases, the pump of the present invention is operable at rotational rates of up to 22,000 rmp. Changing the number and spacing of the discs directly affects the volume, pressure, and ability to pump various types of fluid.

Test Protocols and Results

A 55 gallon drum was fitted with a 1½ inch pipe. This suction line was a 24 inch long fitting over the 1½ inch pipe. The pump inlet was 1¼ to 1½ inches. The pipe outlet on the pump housing matched the port sizes on the baseline pump that was used. A 4 foot column of 1½ inch pipe containing a digital rotary vane flow meter (accuracy of +/−0.5 gpm), a pressure gauge (accuracy +/−¼ psi) was positioned just above the pump, and a ball valve to regulate pressure and a return hose were utilized. No filters were used. Motor type: 230 volt single phase 1.5 HP current rating 7.9 amps, 3450 rpm.

The conventional bladed impeller pump tested had a usable pressure range of 18-24 psi and produced at full flow 6.5 psi @ 93.6 gpm with 6.6 amps. At 18 psi, current was 6.3 amps which consisted of at least 40% volume gases. The working fluid was white and opaque instead of clear. In contrast, the 1.5 HP pump incorporating the impeller assembly of the present invention, at full flow, produced 7.5 psi @ 99.3 gpm with 9.4 amps and the working fluid was visibly clear with no aeration. At the opposite end of the spectrum, when the flow to the conventional bladed impeller pump was restricted, current flow dropped to 4.4 amps (7.9 amp motor rating), which indicates massive aeration. At dead-headed pressure, the pump having the impeller assembly described herein consumed 5.4 amps, indicating that the fluid remains in a normal state for far longer than with the conventional bladed impeller pump. Thus, the rate of failure in stress conditions (low flow) is greatly reduced when using the pump of the present invention.

For a longer test, a 0.5 HP centrifugal pump incorporating an impeller assembly of the present invention was set up in a circulating loop in a 55 gallon drum and left to run for 8 months around the clock. In that time, it pumped 9.3 million gallons at a 120% electrical load with no overheating or malfunctions. The pressure for most of the eight-month test was only 2.45 psi (14% of maximum) and no aeration was observed. The conventional bladed impeller that was tested turned the water completely white when operated at 8.5 psi (47% of maximum), indicating a high level of cavitation, loss of efficiency, and potential damage to the pump.

During long term testing, the water in the drum never exceeded the ambient temperature of 80° F. A conventional bladed impeller pump would have elevated the temperature to at least 120° F. in one day. The water being pumped was unfiltered and contained a variety of particulates that were potential clogging materials. In the 8 month test, the pump of the present invention never lost volume or pressure.

### EXAMPLE 5

### Impeller Assembly Pump for Multi-Stage and Series Centrifugal Pump Applications

A “test pump” incorporating an impeller assembly of the present invention was constructed and, under normal (no flow restrictions) operating conditions produced 6-8 inches of vacuum. When the flow was substantially restricted to 3% of the “normal,” no flow restriction volume, the test pump produced 24 inches of vacuum. When the flow was blocked on the suction side, the test pump produced 27 inches of vacuum. It is anticipated that operation of the test pump at 3% of normal volume could be sustained, producing substantial levels of vacuum and producing high pumping and liquid lifting capacity.

The high vacuum levels observed also indicate that the impeller assembly of the present invention would perform well in multi-stage pump embodiments, as well as in series-pump applications. A multi-stage pump of the present invention may comprise, for example, two or more impeller assemblies driving a common shaft. In a series pump application, multiple pumps, each incorporating one or more impeller assemblies of the present invention, may be assembled, in a series arrangement, to increase the capacity of the system. Additionally, the centrifugal pump incorporating the impeller assembly of the present invention is substantially self-priming and, provided there is liquid in the system, generally does not require a priming operation.

While in the foregoing specification this invention has been described in relation to certain preferred embodiments thereof, and many details have been set forth for purpose of illustration, it will be apparent to those skilled in the art that the invention is susceptible to various changes and modification as well as additional embodiments and that certain of the details described herein may be varied considerably without departing from the basic spirit and scope of the invention.

I claim:

1. A pump system for moving biological fluids comprising:
(a) a housing comprising a first interior chamber, a second interior chamber and a motor housing section;
(b) connections for connecting the first and second interior chambers to at least one body lumen of a patient;
(c) a drive assembly located within the motor housing section; and
(d) a first impeller assembly located within the first interior chamber and a second impeller assembly located within the second interior chamber, wherein each of the first and second impeller assemblies is driven by the drive assembly and comprises:
(i) a hub;
(ii) a stacked array of parallel discs fixedly connected to the hub, wherein each of the discs has a central aperture and an uninterrupted surface area, and wherein the discs are inter-spaced along a parallel axis to form spaces between the discs; and
(iii) a central cavity formed by the central apertures, the central cavity being devoid of a shaft, whereby, upon radial movement of the central hub, fluid is able to flow through the central apertures and through the spaces between the discs.

2. The pump system of claim 1, wherein the drive assembly comprises a magnetic drive system and wherein the hub in each of the first and second impeller assemblies is magnetic.

3. The pump system of claim 2, wherein each of the first and second impeller assemblies further comprises a driving magnet located distal to the hub.

4. The pump system of claim 1, wherein the motor housing is positioned between the first and second interior chambers.

5. The pump system of claim 1, wherein each of the first and second impeller assemblies further comprises a series of spacers, the spacers being fixedly connected to the discs and creating spaces between the discs.

6. The pump system of claim 1, wherein the hub and the stacked array of parallel discs are connected by a plurality of connecting elements.

7. The pump system of claim 6, wherein the connecting elements are connecting rods which are secured to the hub and pass through apertures provided in the discs.

8. The pump system of claim 7, wherein the first and second impeller assemblies rotate on the connecting rods.

9. The pump system of claim 1, wherein the housing is constructed of polycarbonate plastic.

10. The pump system of claim 1, wherein each of the first and second interior chambers is lined with a bio-compatible material that provides an anchoring surface for cells.

11. The pump system of claim 10, wherein the bio-compatible material is a condensation polymer formed from ethylene glycol and terephthalic acid.

12. The pump system of claim 11, wherein the bio-compatible material is Dacron®.

13. The pump system of claim 1, wherein the pump system is a heart pump system, and the connections connect the first and second interior chambers to the patient's cardiovascular system.

14. A filtration device for filtering biological fluids, comprising:
(a) a housing;
(b) a fluid inlet connected to a first body lumen of a patient;
(c) at least one filtering element formed from a semi-permeable membrane for receiving a biological fluid that enters the housing through the fluid inlet;
(d) an impeller assembly for applying pressure to the biological fluid, comprising:
(i) a hub;
(ii) a stacked array of parallel discs fixedly connected to the hub, wherein each of the discs has a central aperture and an uninterrupted surface area, and wherein the discs are inter-spaced along a parallel axis to form spaces between the discs; and
(iii) a central cavity formed by the central apertures, the central cavity being devoid of a shaft, whereby, upon radial movement of the central hub, fluid is able to flow through the central apertures and through the spaces between the discs;
(e) a motor for driving the impeller assembly; and
(f) a fluid outlet connected to a second body lumen of the patient through which the biological fluid exits the housing after passing through the filtering element,
whereby unwanted material is removed from the biological fluid as it passes through the filtering element.

15. A method comprising:
(1) providing a pump system for moving biological fluids, the pump system comprising:
(a) a housing comprising a first interior chamber, a second interior chamber and a motor housing section, each of the first and second interior chambers being lined with a bio-compatible material that provides an anchoring surface for cells;
(b) connections for the first and second interior chambers to at least one body lumen of a patient;
(c) a drive assembly located within the motor housing section; and
(d) a first impeller assembly located within the first interior chamber and a second impeller assembly located with the second interior chamber, wherein each of the first and second impeller assemblies is driven by the drive assembly and comprises:
(i) a hub;
(ii) a stacked array of parallel discs fixedly connected to the hub, wherein each of the discs has a central aperture and an uninterrupted surface area, and wherein the discs are inter-spaced along a parallel axis to form spaces between the discs; and
(iii) a central cavity formed by the central apertures, the central cavity being devoid of a shaft, whereby, upon radial movement of the central hub, fluid is able to flow through the central apertures and through the spaces between the discs; and
(2) culturing the housing with cells for a period of time sufficient to deposit the cells on the lining of the first and second interior chambers.

16. The method of claim 15, further comprising connecting the pump system to the at least one body lumen following deposition of cells on the lining of the first and second interior chambers.

17. The method of claim 15, wherein the pump system is a heart pump system and the cells are vascular cells.

18. The method of claim 15, wherein step (2) comprises circulating media containing the cells through the housing.

19. The method of claim 15, wherein the housing is subjected to an electrical charge of between 0.1 to 10 V during at least a portion of the period of time that the housing is cultured with the cells.

20. The method of claim 15, wherein the bio-compatible material is a condensation polymer formed from ethylene glycol and terephthalic acid.

21. The method of claim 15, wherein the drive assembly comprises a magnetic drive system, the hub in each of the first and second impeller assemblies is magnetic, and each of the first and second impeller assemblies further comprises a driving magnet located distal to the magnetic hub.

22. A pre-conditioned pump system prepared according to the method of claim 15.

23. The pre-conditioned pump system of claim 22, wherein the drive assembly comprises a magnetic drive system, the hub in each of the first and second impeller assemblies is magnetic, and each of the first and second impeller assemblies further comprises a driving magnet located distal to the magnetic hub.

24. A method comprising:
(a) providing an implantable device having an interior lined with a bio-compatible material that provides an anchoring surface for cells; and
(b) circulating media containing the cells through the housing for a period of time sufficient to deposit the cells on the lining.

25. The method of claim 24, wherein the implantable device is subjected to an electrical charge of between 0.1 to 10 V during at least a portion of the period of time that the housing is cultured with the cells.
